(19) United States
(12) Patent Application Publication
(10) Pub. No.: US 2014/0074228 A1
(43) Pub. Date: Mar. 13, 2014

(54) PROSTHETIC HEART VALVE

(71) Applicant: Justin Negri, Armadale (AU)

(72) Inventors: Justin Negri, Armadale (AU); David Husband, Bentleigh (AU)

(73) Assignee: Justin Negri, Armadale (AU)

(21) Appl. No.: 14/018,041

(22) Filed: Sep. 4, 2013

(30) Foreign Application Priority Data
Sep. 4, 2012 (AU) 2012903838

(51) Int. Cl. A61F 2/24

(52) U.S. Cl. CPC A61F 2/2412
USPC 623/2.17

(57) ABSTRACT

A prosthetic mitral valve assembly, comprising a housing in which a plurality of leaflets are pivotally supported, wherein a periphery of the leaflets cooperates with an inner surface of the housing to close the valve and the housing is formed with an external shape which substantially corresponds to that of a mitral valve annulus so as to fit within the annulus.

### FIELD OF THE INVENTION

The present invention relates to a prosthetic heart valve. More particularly, but not exclusively, the present invention relates to a prosthetic mitral valve for use in a human heart.

### BACKGROUND OF THE INVENTION

Biological and mechanical prostheses have been previously proposed for use as replacement heart valves. Although previous biological prosthetic valves provide performance which is comparable to that of a natural human valve, biological valves generally have a limited service life and may require replacement after 10 to 15 years of use.

Previous mechanical prosthetic valves have been found to have a service life far exceeding that of biological valves. However, previous mechanical prosthetic valves have typically been less efficient in some respects and more thrombogenic than biologic valves.

Furthermore, the geometry of previous mechanical valves used for mitral valve replacement, having a circular and planar annulus and having substantially planar leaflets, is different from the natural contour of the mitral annulus in which it is to be received, resulting in a sub-optimal fit of the valve in the annulus which affects the natural blood hemodynamics through and around the valve. Due to the sub-optimal fit, the Tendinous cords and Papillary muscles of the heart require deformation to accept the prosthetic valve and this deformation mimics some disease states and has the potential to impact negatively on function of the heart.

Also, previous mechanical valves having a substantially planar geometry do not take advantage of the available cross sectional of the mitral annulus, thereby creating a flow restriction so that natural flow is impeded, thereby reducing potential performance of the valve compared with a normally functioning natural valve. Previous mechanical valves can also generate a notable acoustic disturbance that can be heard by the patient.

Annuloplasty rings have also been previously used to repair a damaged mitral valve by providing a physical support to the valve. Unlike mechanical prostheses, annuloplasty rings have been configured to follow the natural contours of a mitral annulus so as to support the annulus and assist the natural leaflets of the native valve. Although annuloplasty rings assist the natural function of the heart, they do not address issues arising from damaged leaflets and thus are not useful when a complete mitral valve replacement is required.

Examples of the invention seek to solve, or at least ameliorate, one or more disadvantages of previous prosthetic heart valves.

### SUMMARY OF THE INVENTION

According to one aspect of the present invention, there is provided a prosthetic mitral valve assembly, comprising a housing in which a plurality of leaflets are pivotally supported, wherein a periphery of the leaflets cooperates with an inner surface of the housing to close the valve and the housing is formed with an external shape which substantially corresponds to that of a mitral valve annulus so as to fit within the annulus.

According to preferred embodiments, a cross sectional thickness of the housing is generally constant so that the periphery of the leaflets forms a shape which substantially corresponds to that of the mitral valve. Preferably, the leaflets are configured so as to oscillate around their pivot during flow when the valve is in a fully opened position to facilitate closing of the valve. Preferably, a thickness of each leaflet varies along a longitudinal axis of the leaflet.

According to another aspect of the present invention, there is provided a prosthetic heart valve assembly, comprising a housing in which a plurality of leaflets are pivotally supported, wherein a periphery of the leaflets cooperates with an inner surface of the housing to close the valve and a thickness of each leaflet varies along a longitudinal axis of the leaflet so that the leaflet oscillates around its pivot during flow when the valve is in a fully opened position to facilitate closing of the valve.

Preferred embodiments of the invention further comprise a sewing ring fixed to an outer periphery of housing, the sewing ring being configured to be sewn into the mitral annulus to fix the valve assembly into a patient's heart.

Preferably, said thickness of each leaflet tapers toward a tip of the leaflet. Preferably, a surface of each leaflet which is upstream when the leaflet is in a closed position is not parallel to an opposite surface of the leaflet.

According to preferred embodiments, each leaflet bends through first and second bends as the leaflet extends toward a tip of the leaflet. Preferably, the first bend extends in a direction which is upstream when the valve is in a closed position and the second bend extends in a direction which is downstream when the valve is in a closed position. Preferably, the first bend is in the form of a curve having an axis which is between 2 mm and 10 mm from an axis of rotation of the leaflet, and more preferably between 4 mm and 7 mm.

Preferably, the second bend is in the form of a curve having an axis which is approximately 4 mm from a tip of the leaflet. Preferably, a height of either of the first or second bends is between 0.5 mm and 1.8 mm from a longitudinal axis of the leaflet, and more preferably between 0.85 mm and 1.45 mm.

According to preferred embodiments, a pair of like leaflets are provided, each leaflet being symmetrical about a central plane disposed between the leaflets. A distance from an axis of rotation to a centre of mass of each leaflet can be in the range of 3.0 mm to 5.0 mm and preferably in the range of 3.5 mm to 3.9 mm.

Preferably the assembly is configured so that in a closed position each leaflet is arranged to lie in a plane which is arranged at an angle to a transverse axis of the assembly, the angle being in the range of 22.5 degrees to 47.5 degrees and more preferably in the range of 27 degrees to 33 degrees.

According to another aspect of the present invention, there is provided a leaflet for use in an assembly of the above described type.

### BRIEF DESCRIPTION OF THE DRAWINGS

Preferred embodiments of the invention will be further described, by way of non-limiting example only, with reference to the accompanying drawings in which:

FIG. 1 is a cross sectional view of a human heart;

FIG. 2 is a perspective view of a previous mechanical prosthetic valve;

FIG. 3 is a plan view of the valve of FIG. 2;

FIG. 4 is a cross sectional view of the valve of FIG. 3;

FIG. 5 is a perspective view of a prosthetic mitral valve assembly of one embodiment of the invention;

FIG. 6 is a plan view of the valve assembly;

FIG. 7 is a side sectional view of the valve assembly, the assembly being in a closed condition of use;

FIG. 8 is a side sectional view of the valve assembly, the assembly being in an open condition of use;

FIG. 9 is a perspective view of a leaflet for use in the valve assembly; and

FIG. 10 is a plan view of the leaflet.

### DETAILED DESCRIPTION

A valve assembly 10 according to a preferred embodiment of the present invention is shown in FIG. 5. The valve assembly 10 is configured for use as a prosthetic mitral valve in a human heart.

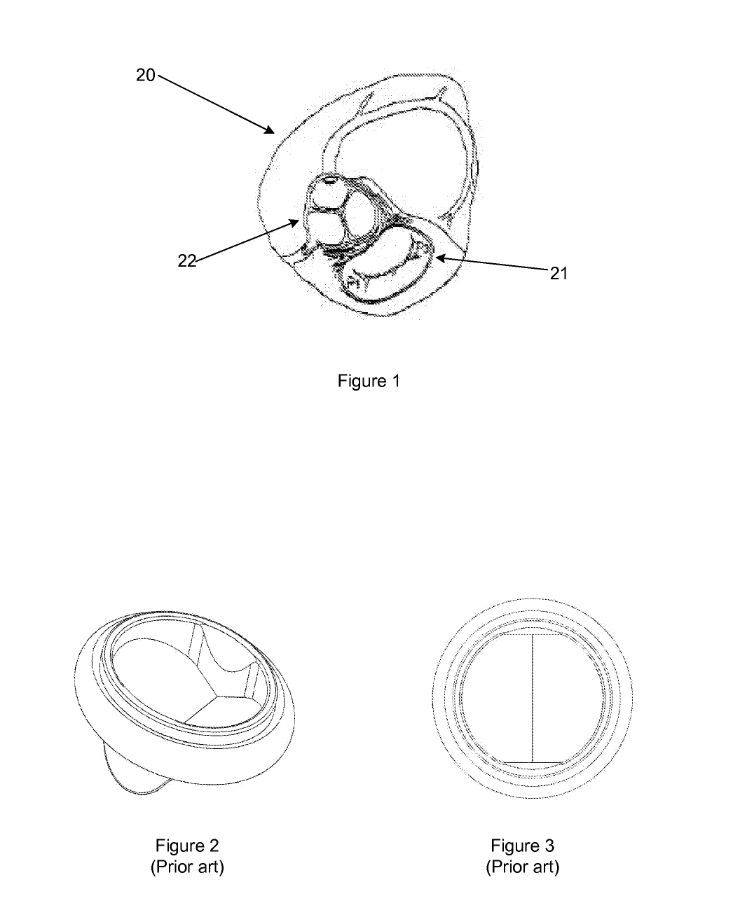

The valve assembly 10 includes a housing 12 in which a plurality of leaflets 14, 16 are pivotally supported. In the described embodiment a pair of like leaflets 14, 16 are provided, although it is envisaged that arrangements having 3 or more leaflets may similarly be provided. In the described embodiment, each leaflet is rigid or substantially rigid and arranged to be symmetrical about a central plane disposed between the leaflets. A periphery of the leaflets 14, 16 cooperates with an inner surface of the housing 12 to close the valve 10. The housing 12 is formed with an external shape which substantially corresponds to that of the mitral annulus 21 (see FIG. 1) so as to fit within the annulus.

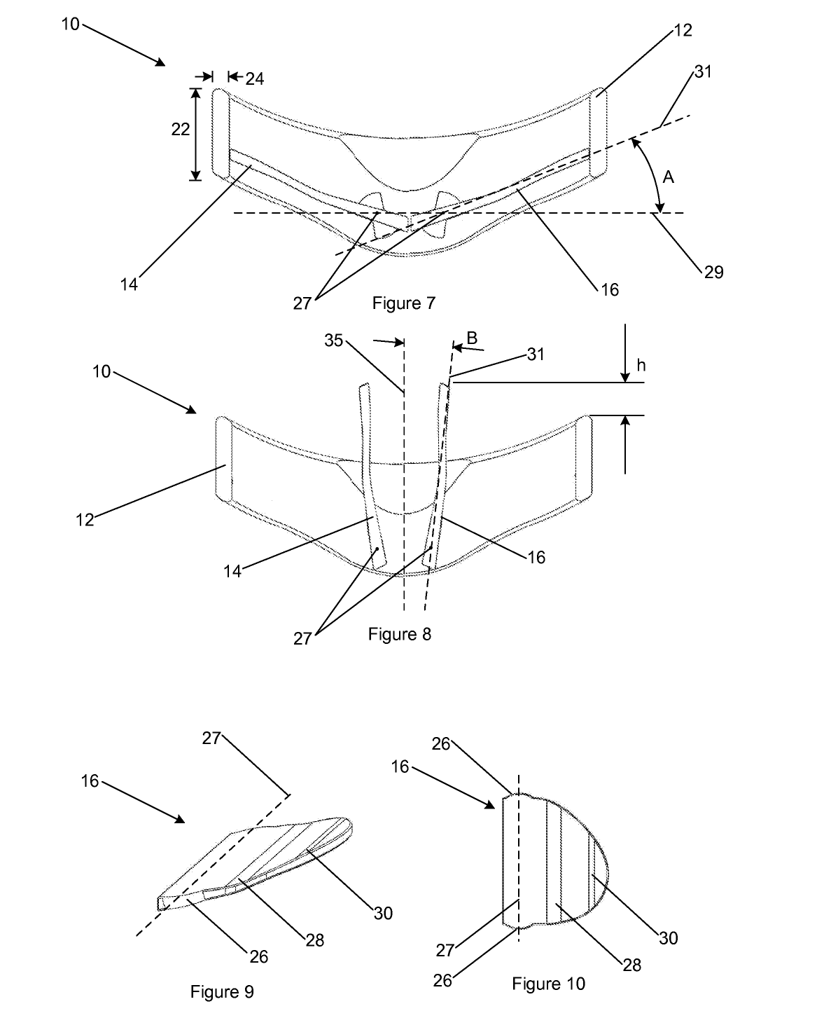

As illustrated in FIGS. 5 and 6, though removed from FIGS. 7 and 8 for clarity, the assembly 10 also includes a sewing ring 18 which is fixed to an outer periphery of housing 12. The sewing ring 18 is configured to be sewn into the mitral annulus 21 of a patient to fix the valve assembly into a patient's heart.

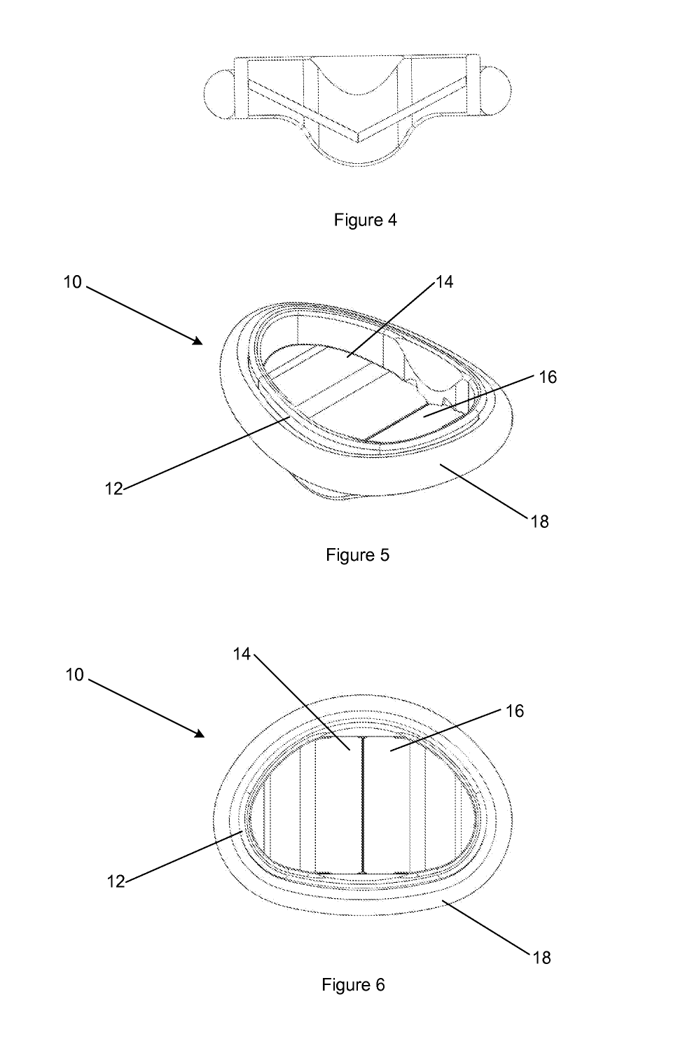

As can be seen in FIG. 1, the mitral valve annulus 21 of a human heart 20 generally has an irregular bean-shape in plan view. As illustrated in FIG. 6, an outer periphery of assembly 10 also has a generally irregular bean-shape so as to be complimentary with the annulus. The housing 12 is also non-planar and varies in three dimensions so as to be non-constant in three orthogonal planes and complimentary with the contours or profile of the mitral annulus 21, which has a generally three dimensional saddle shape. Previous mechanical prostheses, such as those shown in FIGS. 2, 3 and 4, have been circular in cross sectional shape and essentially planar, generally in accordance with an aortic annulus 22 as shown in FIG. 1, so that a patient's heart is required to be deformed to accept the valve, thereby placing undue strain on the heart.

Because the housing 12 of the assembly 10 is formed so as to have a shape which corresponds with that of a natural mitral annulus 21 of the heart 20, the valve assembly 10 may be fitted to the heart of a patient generally without deforming or placing undue stress on it. Furthermore, the housing 12 acts to support the annulus 21 in the same way an annuloplasty ring does, thereby allowing the valve assembly 10 to address problems associated with damaged leaflets and also damage to the annulus 21 with a single device.

As can be seen in FIG. 6, a periphery of the leaflets 14, 16 cooperates with an inner surface of the housing 12 to close the valve 10. Although the shape of the housing 12 varies in three dimensions, a cross sectional thickness 24 of the housing 12, as shown in FIG. 7, is generally constant. A depth 22 of the housing 12 may also be constant. As the cross sectional thickness 24 is generally constant, the periphery of the leaflets 14, 16 forms a shape which generally corresponds to that of the mitral annulus, thereby taking full advantage of an available cross sectional flow area and increasing the cross sectional flow area of the valve over conventional circular valves when opened to address flow restrictions of previous circular valves.

It will be appreciated that increasing the size of the leaflets increases the sealing area between the larger leaflets and the housing 12, which can lead to increases in the regurgitant volume or flow-back of the valve on closing. To address this issue, the hydrodynamic properties of the leaflets 14, 16 have been modified to improve their opening and closing performance to reduce regurgitant volume of the valve assembly.

One feature of the leaflets 14, 16 which acts to improve the opening and closing performance is that they are configured so as to have increased sensitivity to radial acceleration during flow when the valve is in a fully opened position. In this regard, the leaflets oscillate or flutter during flow and may be considered to be hydrodynamically unstable, i.e., continues to move during steady flow. It has been found, using computational fluid dynamic analysis, that increasing the sensitivity to radial acceleration of the leaflets 14, 16 when in the open position leads to enhanced acceleration of the leaflet during closing. This facilitates closing of the valve by improving the initiation of movement of the valve from the fully opened position.

Previously, oscillation or flutter of the leaflets has been considered to be a negative attribute as high speed impact between a leaflet and blood flowing through the valve is thought to cause damage to the blood. This is particularly true for aortic valves, however, as the flow of blood through a mitral valve is of a lower velocity, it is now believed that any damage caused is negligible and thus a less important consideration, thereby allowing a small amount of instability to be used to improve the hydrodynamics of the leaflets to reduce the regurgitant volume of the valve.

In preferred embodiments, each of the leaflets 14, 16 is curved in profile to generate a controlled amount of oscillation. Shear stresses acting on the curved surfaces of each leaflet by blood flowing over each leaflet results in forces acting upon the leaflet and causes movement, thereby making the leaflet oscillate around the pivot in use.

In some embodiments, a surface of each leaflet which is upstream when the leaflet is in a closed position is not parallel to an opposite surface of the leaflet. The opposite surfaces of the leaflets 14, 16 are not parallel so that forces acting on opposite sides of the leaflets 14, 16 are not matched in location with respect to a centre of mass of a leaflet, thereby causing movement in the form of oscillation or flutter of the leaflet.

As illustrated in FIGS. 7 and 8, a thickness of each leaflet 14, 16 varies along a longitudinal axis of the leaflet. In some embodiments, the thickness of each leaflet tapers toward a tip of the leaflet. In other embodiments, the leaflets may taper toward a tip of the leaflet without being curved. Varying the thickness of each leaflet allows adjustment of the centre of mass so that the rotational inertia of the leaflet can be varied to control the sensitivity of the leaflet to hydrodynamic forces acting upon it. In this regard, the magnitude of radial acceleration of the leaflet can be varied in response to hydrodynamic forces acting upon it.

In preferred embodiments, each leaflet 14, 16 bends through first and second bends 28, 30 as the leaflet extends toward a tip of the leaflet. The first bend 28 extends in a direction which is upstream when the valve is in a closed position and the second bend 30 extends in a direction which is downstream when the valve is in a closed position. The first bend 28 is generally in the form of a curve having an axis which is located between 2 mm and 10 mm, and preferably between 4 mm and 7 mm, from an axis of rotation 27 of the leaflet. The first bend 28 extends into the path of fluid flowing over the leaflet and causes forces to act on the leaflet, thereby inducing movement. A rear or concaved side of the first bend 28 is provided to reduce localised pressure during flow and promote a tendency for the valve to move toward the closed position. The position of the first bend has been selected to so as to control the location of forces acting on the leaflet so as to control the magnitude of oscillation induced in the leaflet.

The second bend 30 is in the form of a curve having an axis which is approximately 4 mm from a tip of the leaflet. The second bend 30 is formed at this position to return the tip of the leaflet in the direction of preferred flow and promote laminar fluid flow through the valve. A rear or concaved side of the second bend 30 acts to induce forces on the leaflet which act against forces on the opposite side of the leaflet to cause oscillation.

Importantly, a height of either of the first or second bends 28, 30 is such as to create a controlled amount of sensitivity of the leaflet without causing excessive turbulence so as to be detrimental to the flow of blood through the assembly 10. Accordingly, the depth of curvature has been carefully selected so as not to create eddies in the blood flow. In this regard, a height of either of the first or second bends is between 0.5 mm and 1.8 mm, and preferably between 0.85 mm and 1.45 mm, from a longitudinal axis 31 of the leaflet.

As illustrated in FIG. 10, each leaflet 14, 16 is pivotally supported within the housing 12 by way of mounting lugs 26 formed on sides of each leaflet so that they can pivot about axis 27. In the described embodiment, each leaflet 14, 16 tapers in the region of the pivot axis 27 and the lugs 26 are also tapered so as to maximise laminar flow over each leaflet surface. The location of lugs 26 dictates the location of the pivot axis 27 and its spatial relationship with a centre of mass of the leaflet. The location of the lugs 26 also dictates the separation of respective pivot axes 27 of each leaflet. The location of the pivot axis 27 is also chosen such that a length of each leaflet 14, 16 is restricted so that a tip of the leaflet does not extend too far from the housing 12 (see height h in FIG. 8).

A distance from the axis of rotation 27 to a centre of mass of each leaflet is in the range of 3.0 mm to 5.0 mm and is preferably in the range of 3.5 mm to 3.9 mm. It will be appreciated that moving the location of the centre mass towards the axis 27 will increase the radial acceleration of the leaflet upon closing, promoting a reduction in regurgitant volume of the valve and also notably increase the magnitude and/or frequency of oscillation.

Accordingly to preferred embodiments of the present invention, varying the leaflet thickness to control the location of the centre of mass, in combination with the curved profile design achieves a balanced amount of sensitivity that provides an increase in radial acceleration for improving closing response of the valve whilst controlling the amount of flutter at full flow, and still maintaining a design capable of cost effective manufacture using convention materials and processes. In this regard, the housing and leaflets of the described heart valve may be manufactured using conventional moulding processes and formed of pyrolytic coated graphene, titanium or medical grade polymers and the sewing ring from medical grade polypropylene, for example. The amount of oscillation induced is believed to be small so as to maintain substantially laminar flow of blood upon departure of the leaflet tip and minimise turbulent flow so as to maximise flow volume. Ideally, oscillation is only induced in the leaflets as flow decreases, i.e. immediately prior to closing of the valve.

FIG. 7 illustrates the valve assembly 10 in a closed position. The assembly 10 is configured so that in a closed position a longitudinal axis 31 of each leaflet 14, 16 is arranged at an angle A to a transverse axis 29 of the assembly. In the described embodiment, the angle A is in the range of 22.5 degrees to 47.5 degrees and is preferably in the range of 27 degrees to 33 degrees. Previous mechanical prosthetic valves have utilised a smaller closing angle so that leaflets lie in a plane which is closer to a transverse axis of the valve, however, increasing the closing angle has the benefit of reducing the leaflet closing velocity, potentially reducing impact of the leaflets against the housing and thereby reducing the audible volume of noise created upon closing of the valve. Furthermore, increasing the closing angle allows the valve assembly 10 to close faster, thereby further reducing the regurgitant volume.

FIG. 8 illustrates the valve assembly 10 in an open position. The assembly 10 is configured so that in the open position a longitudinal axis 31 of each leaflet is arranged at an angle B to a longitudinal axis 35 of the assembly, the angle B being in the range of 80 to 86 degrees and preferably approximately 84.5 degrees.

The leaflets 14, 16 have been described as cooperating with an inner surface of the housing 12 to close the valve assembly 10. It will be appreciated that the leaflets do not need to contact the housing 12 to achieve this and that a liner or other intermediate component may be provided between the leaflets 14, 16 and the housing 12 to further seal the valve assembly 10.

While the preferred embodiments have been described in relation to a prosthetic mitral valve, those skilled in the art will appreciate that the described leaflets may provide superior performance to previous leaflets and thus have application in circular prosthetic valves, such as those used for aortic valve replacement. Accordingly, in another embodiment the prosthetic valve assembly (not shown) includes a housing in which a plurality of leaflets are pivotally supported, wherein a periphery of the leaflets cooperates with an inner surface of the housing to close the valve and a thickness of each leaflet varies along a longitudinal axis of the leaflet so that the leaflet oscillates around its pivot during flow when the valve is in a fully opened position to facilitate closing of the valve.

In such an embodiment, the leaflets may be similarly configured to those described above, however, the magnitude of oscillation generated in the leaflets may be reduced to accommodate the high velocity of blood flowing through the valve. This may be achieved by reducing the amount of variation in thickness of the leaflets, reducing the height of bends formed in the leaflets or by modifying the centre of mass to vary the sensitivity of the leaflets to these design parameters.

The described prosthetic valves have been described having regard to the heart of a patient in which it is to be received. It will be appreciated that differently sized prostheses may be required. Accordingly, the described valves may be provided in a number of different sizes which correspond to predetermined size ranges of patients.

While the preferred embodiments of the invention have been described in relation to a human heart, it will also be appreciated that the invention will have application to animal hearts also.

The embodiments have been described by way of example only and modifications are possible within the scope of the invention disclosed.

1. A prosthetic mitral valve assembly, comprising a housing in which a plurality of leaflets are pivotally supported, wherein a periphery of the leaflets cooperates with an inner surface of the housing to close the valve and the housing is formed with an external shape which substantially corresponds to that of a mitral valve annulus so as to fit within the annulus.

2. An assembly according to claim 1, wherein a cross sectional thickness of the housing is generally constant so that the periphery of the leaflets forms a shape which substantially corresponds to that of the mitral valve.

3. An assembly according to claim 1, wherein the leaflets are configured so as to oscillate around their pivot during flow when the valve is in a fully opened position to facilitate closing of the valve.

4. An assembly according to claim 1, wherein a thickness of each leaflet varies along a longitudinal axis of the leaflet.

5. A prosthetic heart valve assembly, comprising a housing in which a plurality of leaflets are pivotally supported, wherein a periphery of the leaflets cooperates with an inner surface of the housing to close the valve and a thickness of each leaflet varies along a longitudinal axis of the leaflet so that the leaflet oscillates around its pivot during flow when the valve is in a fully opened position to facilitate closing of the valve.

6. An assembly according to claim 1, further comprising a sewing ring fixed to an outer periphery of housing, the sewing ring being configured to be sewn into the mitral annulus to fix the valve assembly into a patient's heart.

7. An assembly according to claim 1, wherein said thickness of each leaflet tapers toward a tip of the leaflet.

8. An assembly according to claim 1, wherein a surface of each leaflet which is upstream when the leaflet is in a closed position is not parallel to an opposite surface of the leaflet.

9. An assembly according to claim 1, wherein each leaflet bends through first and second bends as the leaflet extends toward a tip of the leaflet.

10. An assembly according to claim 9, wherein the first bend extends in a direction which is upstream when the valve is in a closed position and the second bend extends in a direction which is downstream when the valve is in a closed position.

11. An assembly according to claim 9, wherein the first bend is in the form of a curve having an axis which is between 2 mm and 10 mm from an axis of rotation of the leaflet.

12. An assembly according to claim 11, wherein the first bend is in the form of a curve having an axis which is between 4 mm and 7 mm from an axis of rotation of the leaflet.

13. An assembly according to claim 9, wherein the second bend is in the form of a curve having an axis which is approximately 4 mm from a tip of the leaflet.

14. An assembly according to claim 9, wherein a height of either of the first or second bends is between 0.5 mm and 1.8 mm from a longitudinal axis of the leaflet.

15. An assembly according to claim 14, wherein said height is between 0.85 mm and 1.45 mm.

16. An assembly according to claim 1, wherein a pair of like leaflets are provided, each leaflet being symmetrical about a central plane disposed between the leaflets.

17. An assembly according to claim 1, wherein a distance from an axis of rotation to a centre of mass of each leaflet is in the range of 3.0 mm to 5.0 mm.

18. An assembly according to claim 17, wherein the distance is in the range of 3.5 mm to 3.9 mm.

19. An assembly according to claim 1, being configured so that in a closed position each leaflet is arranged to lie in a plane which is arranged at an angle to a transverse axis of the assembly, the angle being in the range of 22.5 degrees to 47.5 degrees.

20. An assembly according to claim 19, wherein the angle is in the range of 27 degrees to 33 degrees.

21. A leaflet for use in an assembly according to claim 1.
